(19) United States
(12) Patent Application Publication
(10) Pub. No.: US 2026/0224552 A1
(43) Pub. Date: Aug. 6, 2026

(54) HYDROGELS INCORPORATING ATROPINE

(71) Applicant: UNIVERSIDADE DE SANTIAGO DE COMPOSTELA, Santiago de Compostela (ES)

(72) Inventors: Carmen Isabel ÁLVAREZ LORENZO, Santiago de Compostela (ES); Inés GARCÍA VALLE, Santiago de Compostela (ES); María VIVERO LÓPEZ, Santiago de Compostela (ES); Angel CONCHEIRO NINE, Santiago de Compostela (ES)

(21) Appl. No.: 19/149,602

(22) PCT Filed: Jan. 22, 2024
(86) PCT No.: PCT/EP2024/051379

§ 371 Date: Jul. 21, 2025

(30) Foreign Application Priority Data
Jan. 23, 2023 (EP) 23382050.5

(51) Int. Cl. A61K 31/46; A61K 9/06; A61K 47/32

(52) U.S. Cl. CPC A61K 31/46; A61K 9/06; A61K 47/32

(57) ABSTRACT

The invention relates to acrylic hydrogels comprising the myopia-suppressing agent atropine, and to methods for preparing the same. The hydrogels are capable of loading high quantities of atropine and releasing it in a sustained manner over an extended period of time suitable for overnight use.

### FIELD OF THE INVENTION

This invention relates to the field of hydrogels. More particularly, the invention relates to acrylic hydrogels which incorporate atropine and to methods for preparing such hydrogels.

### BACKGROUND

Myopia, also known as nearsightedness, is a very common vision disorder affecting distance vision. It is estimated that the number of people affected by it will reach fifty percent of the global population by 2050, due mainly to lifestyle factors.

This condition has been the subject of extensive research for decades. Several interventions to decrease the progression of myopia have been proposed and investigated, including devices that alter visual perception and pharmacological treatments. Among the latter, atropine is currently the most promising drug for myopia control.

Atropine is typically administered via eye drop instillation. However, each drop is only present on the eye for a very short time, since most of the drop either spills out of the eye or gets washed down the tear drainage pathway into the nose and throat. As a result, only a minimal amount of drug reaches the ocular tissues, while the rest may be absorbed systemically with potential side effects. In summary, atropine eye drop treatment leads to a peak-trough drug concentration that fails to deliver a sustained and effective, yet tolerable, dose into the eye for myopia progression control. Consequently, ophthalmic drop treatment is generally a very poor way to administer atropine to the eye.

As an alternative, contact lenses (CLs) have long been proposed as an appealing system for continuous delivery of ophthalmic drugs. However, incorporating atropine into CLs has proven to be challenging, due to the particular characteristics of the therapeutically used form of atropine, i.e., atropine sulfate. This molecule forms a complex between two atropine molecules interacting with a single sulfate ion. Hence, the salt has a considerably higher molecular weight and bulk and hydrophilic character than other ophthalmic drugs. Furthermore, due to the restrictive requirements of CL in terms of comfort, optical quality and oxygen permeability, the available choices of material and tailoring of material chemistry for CLs are rather limited.

Two main strategies have been attempted in the prior art when it comes to loading the atropine into the CL, namely immersing a previously formed CL in a solution comprising the atropine; or polymerizing monomers in the presence of atropine to directly form the atropine loaded CL.

The earlier method is preferred since loading by immersion means that the CL can be reloaded with atropine after usage as many times as is desired.

However, solutions based on the above methods have so far shown opposite, extreme behaviors.

Attempts based on the earlier method have led to CLs providing an immediate release of atropine. Atropine has low affinity to interact with the CL polymer network, remaining preferentially in the aqueous phase of the hydrogel, and drug diffusion into the network is hampered by the large size of the molecule. This leads to a poor, mainly superficial, drug charge that is rapidly discharged upon contact with the tear fluid (burst effect).

Hui et al. In vitro release of two anti-muscarinic drugs from soft contact lenses. Clinical Ophthalmology, 11, (2017), 1657; evaluated atropine release from numerous commercially available CLs based on structurally differing hydrogel materials into which the atropine sulfate had been loaded by immersion. For all the CLs tested, a marked immediate release of atropine was observed.

On the other hand, the latter method (polymerization in the presence of atropine) has led to CLs which release atropine in an excessively controlled (i.e. slow) manner, in an order of months, since the atropine sulfate is highly and deeply entangled within the hydrogel cross-linked network.

Lasowski et al. Atropine and Roscovitine Release from Model Silicone Hydrogels. Optometry and Vision Science, 9, (2016), 404; examined atropine delivery from hydrogel materials comprising N,N-Dimethylacrylamide and the silicone monomer 3-Methacryloxypropyltris(trimethylsiloxy)silane (TRIS) or TRIS-OH. Atropine was directly loaded into the hydrogel materials before polymerization of the hydrogel network. Although the authors observed no degradation of the drug due to UV exposure or heat treatment during polymerization, the potential problems derived from low stability of atropine at the high temperatures required for CL sterilization have not been taken into account. In addition, this approach results in a non-reusable system where a considerable amount of the drug remains in the material due to irreversible trapping of atropine in poorly hydrated silicone domains formed during synthesis.

Therefore, there exists a need to develop hydrogel compositions with enhanced atropine loading capacity that, in turn, provide an effective means of delivering an efficacious and safe amount of atropine over an extended period of time of several hours. Such systems would be particularly beneficial for overnight delivery of atropine. Furthermore, the hydrogel should ideally be re-loadable, enabling multiple uses of a same CL. CLs based on such compositions would represent a more effective therapeutic tool and potentially enhance patient compliance.

### BRIEF DESCRIPTION OF THE INVENTION

The present inventors have now surprisingly found that with the hydrogels according to the invention, it is possible to prepare contact lenses that incorporate therapeutic amounts of atropine sulfate by immersion in a solution of atropine sulfate. Additionally, the hydrogels of the present invention unexpectedly show a sustained release profile of atropine sulfate when applied to the ocular surface for a prolonged period, such as of 6 to 10 hours, particularly useful for overnight contact lens use.

Thus, in a first aspect, the invention is directed to a hydrogel comprising:
a polymer network, wherein the polymer network:
   comprises acrylic monomeric units in an amount of at least 90% by weight with respect to the total weight of the polymer network; and
   is cross-linked;
atropine,
wherein the acrylic monomeric units comprised in the polymer network comprise anionic acrylic monomeric units; and
wherein the acrylic monomeric units comprised in the polymer network comprise aromatic acrylic monomeric units and/or comprise silicon oxide acrylic monomeric units, preferably
wherein the aromatic groups comprised in said aromatic acrylic monomeric units comprise no N heteroatoms, more preferably comprise no N, O or S heteroatoms, even more preferably comprise no heteroatoms.

A second aspect of the present invention relates to a hydrogel precursor comprising:
a polymer network, wherein the polymer network:
   comprises acrylic monomeric units in an amount of at least 90% by weight with respect to the total weight of the polymer network; and
   is cross-linked;
wherein the acrylic monomeric units comprised in the polymer network comprise anionic acrylic monomeric units; and
wherein the acrylic monomeric units comprised in the polymer network comprise aromatic acrylic monomeric units and/or comprise silicon oxide acrylic monomeric units, preferably wherein the aromatic groups comprised in said aromatic acrylic monomeric units comprise no N heteroatoms, more preferably comprise no N, O or S heteroatoms, even more preferably comprise no heteroatoms.

A third aspect of the present invention relates to a method for preparing a hydrogel, comprising the steps of:
a) Providing a precursor hydrogel comprising a polymer network, wherein the polymer network:
   comprises acrylic monomeric units in an amount of at least 90% by weight with respect to the total weight of the polymer network, wherein said acrylic monomeric units comprise anionic acrylic monomeric units; and wherein said acrylic monomeric units comprise aromatic acrylic monomeric units and/or silicon oxide acrylic monomeric units, preferably wherein the aromatic groups comprised in said aromatic acrylic monomeric units comprise no N heteroatoms, more preferably comprise no N, O or S heteroatoms, even more preferably comprise no heteroatoms;
   is cross-linked;
b) Providing an atropine solution;
c) Combining the precursor hydrogel of step a) and the atropine solution of step b).

Thus, additionally or alternatively to the above definition characterising the hydrogels of the invention, a hydrogel of the first aspect of the present invention can be characterised as one which is obtainable by a method according to the third aspect of the invention.

An additional aspect of the present invention relates to the hydrogels of the present invention for use in medicine.

### BRIEF DESCRIPTION OF THE DRAWINGS

FIG. 1. Atropine loaded by precursor hydrogels B2x, B3x, S2b, S3b, S2x and S3x after immersion in 5 mL of a solution of atropine sulfate (1 mg/mL) prepared in distilled water or in NaCl 0.9%, at 25° C. and 180 rpm for 24 h. Mean values and standard deviations (n=4).

FIG. 2. Atropine release profiles recorded for hydrogel discs B2x, B3x, S2b, S3b, S2x and S3x expressed as percentage in 6 mL of NaCl 0.9% solution for 72 h at 36° C. and 180 rpm. Mean values and standard deviations (n=4).

FIG. 3. Atropine release profiles recorded for sterilized CLs prepared from hydrogels B3x and S2x and commercially available Acuvue Moist and Proclear CLs, after loading in 0.2 mg/mL atropine sulfate solution, expressed as (A) amount released and (B) percentage released in 6 mL of NaCl 0.9% solution at 36° C. and 180 rpm. Mean values and standard deviation (n=4).

FIG. 4. In vivo tear fluid levels of atropine recorded after instillation of one single drop (50 μL) of 0.2 mg/mL atropine sulfate in NaCl 0.9% sterilized solution and during wearing of sterilized atropine-loaded S2x CLs for 6 h. Mean values and standard deviations (n=4).

FIG. 5. Levels of atropine in rabbit eye tissues recorded 6 h after instillation of one single drop of sterilized atropine solution (50 μL, 0.2 mg/mL in NaCl 0.9%) and after wearing of sterilized atropine-loaded S2x CLs for 6 h for (A) aqueous humor in right eyes (AHR), vitreous humor in right eyes (VHR), aqueous humor in left eyes (AHL), and vitreous humor in left eyes (VHL); (B) cornea, retina and sclera recorded in right eyes (R); and (C) cornea, retina and sclera recorded in left eyes (L). The treatment was applied on the right eyes, while left eyes were used as controls (without treatment; stripped bars). The bars represent mean values and standard deviations (n=4).

### DETAILED DESCRIPTION OF THE INVENTION

In a first aspect, the present invention is directed to a hydrogel as was described above.

The term “hydrogel” refers to a three-dimensional network of polymer chains which is capable of absorbing and retaining water to form a gel in which water is the dispersion medium. Thus, a hydrogel comprises a polymer network and water, and in the context of the present invention may further comprise atropine (depending on whether the precursor hydrogel or the final hydrogel is being referred to). However, weights described herein referring to the polymer network and its monomeric units refer to the weight of solely these components and do not comprise weights of other hydrogel components such as water or atropine.

In an embodiment, the hydrogels of the present invention comprise water in an amount of from 20 to 80% by weight with respect to the total weight of the hydrogel.

In an embodiment, the size of the shortest dimension of the hydrogels of the present invention is greater than 1 μm. Preferably, the size of the shortest dimension of the hydrogels of the present invention is at least 5 μm, more preferably at least 10 μm, even more preferably at least 50 μm, such as from any of these values up to 10 cm, preferably up to 1 cm, more preferably up to 200 μm. Additionally or alternatively, the hydrogels of the present invention possess a dimension sized at least 1 mm, preferably at least 5 mm, more preferably at least 9 mm, such as from any of these values up to 100 cm, preferably up to 10 cm, more preferably up to 16 mm.

In a particularly preferred embodiment, the size of the shortest dimension of the hydrogels of the present invention is between 50 and 200 μm, and the hydrogels possess a dimension sized between 9 and 16 mm.

The polymer network comprised in the hydrogels of the present invention comprises acrylic monomeric units in an amount of at least 90% by weight with respect to the total weight of the polymer network. Preferably, said amount is at least 95%; more preferably said amount is at least 99%; and even more preferably, the polymer network does not comprise any monomeric unit different to acrylic monomeric units. In an embodiment, the polymer network consists of acrylic monomeric units. The hereinbelow amounts of the different individual acrylic monomeric units with respect to the total weight of acrylic monomeric units apply regardless of the percentage which said total acrylic monomeric units represent with respect to the total weight of the polymer network.

The term “monomeric unit”, or “unit” (in the context of polymers), or “repeating unit”, refers to the structural motif in a polymer that stems from a monomer that has been subjected to polymerization. It therefore does not include any non-polymerizable compound which many end up in a non-recurring manner in a polymer chain, such as initiator molecules. It is distinguished from the monomer in that it is part of the polymer, whereas a monomer is an independent molecular entity which can be polymerized into a polymer. It is commonplace in the art to refer to monomeric units according to the structure of the monomer, even though the monomeric unit itself may no longer show exactly the same structure as the monomer. Thus, for instance, “acrylic monomeric unit” actually refers to a monomeric unit derived from an acrylic monomer by polymerization, even though the acrylic monomeric unit no longer comprises the C═C double bond of the acrylic group that was present in the monomer, as this is the bond which has enabled the polymerization to procced, i.e. reacted with the incoming initiator or growing polymeric chain and then with further monomers. Similarly, ethylene oxide monomeric units do not actually comprise ethylene oxide epoxide, but refer to the unit resulting from its polymerization. The skilled person is well aware of which monomers correspond to which monomeric units. Similarly, the skilled person is well aware of how to convert monomers into corresponding monomeric units by a process of polymerization.

When the present invention refers to a monomeric unit comprising a functional group, said unit is termed [functional group] monomeric unit. For example, an acrylic monomeric unit further comprising an anionic group is herein termed an anionic acrylic monomeric unit.

The term “polymer”, also identified by the prefix “poly”, herein refers to a molecule comprising at least 10 monomeric units, such as at least 100 or at least 1000 monomeric units. Unless otherwise indicated, poly[monomeric unit X], herein refers to a molecule comprising at least 10 X monomeric units, such as at least 100 or at least 1000 X monomeric units, wherein monomeric unit X refers to a specific monomeric unit.

As used herein, the term “acrylic monomer” refers not only to acrylic acid or acrylic acid ester monomers, but also to alkylacrylic acid or alkylacrylic acid ester monomers, such as methacrylic acid or methacrylic acid ester monomers, and optionally also to vinylcarbonate analogues thereof wherein an oxygen atom is found between the carbonyl and C═C double bond group of the acrylic acid ester or alkylacrylic acid ester monomer, such as poly(dimethylsiloxy)di(silylbultanol)bis(vinyl carbamate). As used herein, the term “acrylic monomer” refers also to acrylamide and alkylacrylamide monomers such as methacrylamide monomers, and optionally also to vinylcarbamate analogues thereof wherein an oxygen atom is found between the carbonyl and C═C double bond group of the acrylamide or alkylacrylamide monomer, such as 3-[Tris(trimethylsiloxy)silyl]propyl vinyl carbamate. Preferably, the C═C double bond in the acrylic monomer is a terminal C═C double bond. The term “alkyl” refers to a straight or branched fully saturated hydrocarbon group. The alkyl is preferably a C₁₋₁₂ alkyl; more preferably a C₁₋₆ alkyl; even more preferably a methyl, ethyl or propyl; most preferably it is a methyl; group.

In an embodiment, the acrylic monomeric units comprised in the polymer network comprised in the hydrogels of the present invention comprise monofunctionalized acrylic monomeric units. A monofunctionalized acrylic monomeric unit stems from a monomer containing a single acrylic group. More particularly, a polymer network comprising monofunctionalized acrylic monomeric units is the result of the polymerization of a monomer mixture comprising monomers containing a single acrylic group. In a preferred embodiment, the acrylic monomeric units comprise an amount of at least 90%; preferably of at least 99.0%; more preferably of at least 99.6%; by weight of monofunctionalized acrylic monomeric units with respect to the total weight of acrylic monomeric units. In any embodiment described herein, the maximum possible amount by weight of monofunctionalized acrylic monomeric units with respect to the total weight of acrylic monomeric units is dictated by the amount of bifunctionalized acrylic monomeric units required by said embodiment. Thus, at least 90% by weight of monofunctionalized acrylic monomeric units becomes 90% to 99.90% by weight of monofunctionalized acrylic monomeric units if the embodiment requires 0.10% by weight of bifunctionalized acrylic monomeric units.

Non-limiting examples of monofunctionalized acrylic monomeric units are 2-hydroxyethyl methacrylate (HEMA), N-(3-aminopropyl) methacrylamide (APMA), N,N-dimethylacrylamide, N,N-diethylacrylamide, methyl methacrylate or cyclohexyl methacrylate, or combinations thereof.

Preferably, the monofunctionalized acrylic monomeric units comprise alkyl or alkanol acrylic monomeric units, i.e. units wherein the non-carbonyl ester oxygen or the amide nitrogen of the acrylic group is attached to an alkyl or alkanol group, wherein the term “alkyl” is as defined above, and the term “alkanol” refers to an alkyl group as defined above substituted with an —OH group, such as HEMA monomeric units. More preferably, the monofunctionalized acrylic monomeric units comprise HEMA monomeric units. Even more preferably, at least 35%, preferably at least 85%, by weight of the monofunctionalized acrylic monomeric units, which are preferably in the above stated amounts, are alkyl or alkanol acrylic monomeric units, preferably HEMA monomeric units. The alkyl group of alkyl acrylic monomers is not to be confused with the alkyl group of alkylacrylic monomers. In alkylacrylic monomers, the alkyl group is connected to the C═C bond of the acrylic group. Thus, alkyl alkylacrylic monomers are common in the art, such as ethyl methacrylate (compound with the formula C₂H₅O(C═O)C(CH₃)═CH₂).

In an embodiment, the acrylic monomeric units comprised in the polymer network comprised in the hydrogels of the present invention comprise bifunctionalized acrylic monomeric units. A bifunctionalized acrylic monomeric unit stems from a monomer containing two or more acrylic groups. More particularly, a polymer network comprising bifunctionalized acrylic monomeric units is the result of the polymerization of a monomer mixture comprising monomers containing two or more acrylic groups. In a preferred embodiment, the acrylic monomeric units comprise an amount of between 0.01% and 10% by weight of bifunctionalized acrylic monomeric units with respect to the total weight of acrylic monomeric units; preferably said amount is of between 0.10% and 1.0%, more preferably said amount is of between 0.15% and 0.4%, by weight of bifunctionalized acrylic monomeric units with respect to the total weight of acrylic monomeric units.

Preferably, the acrylic groups in the bifunctionalized acrylic monomeric unit are connected through an alkyl linker, wherein the term “alkyl” is as defined above. More preferably, the non-carbonyl ester oxygen or the amide nitrogen of an acrylic group is connected through an alkyl linker to the non-carbonyl ester oxygen or to the amide nitrogen of another acrylic group.

Non-limiting examples of bifunctionalized acrylic monomeric units are ethylene glycol dimethacrylate (EGDMA), 1,3-butanediol diacrylate, 1,4-butanediol diacrylate, 1,6-hexanediol diacrylate, ethylene glycol diacrylate, fluorescein O,O′-diacrylate, glycerol 1,3-diglycerolate diacrylate, pentaerythritol diacrylate monostearate, 1,6-hexanediol ethoxylate diacrylate, 3-hydroxy-2,2-dimethylpropyl 3-hydroxy-2,2-dimethylpropionate diacrylate, bisphenol A ethoxylate diacrylate, di(ethylene glycol) diacrylate, neopentyl glycol diacrylate, propylene glycol glycerolate diacrylate, tetra(ethylene glycol) diacrylate, 1,3-butanediol dimethacrylate, 1,4-butanediol dimethacrylate, 1,6-hexanediol dimethacrylate, bisphenol A dimethacrylate, diurethane dimethacrylate, fluorescein O,O′-dimethacrylate, glycerol dimethacrylate, bisphenol A ethoxylate dimethacrylate, bisphenol A glycerolate dimethacrylate, di(ethylene glycol) dimethacrylate, tetraethylene glycol dimethacrylate, tri(ethylene glycol) dimethacrylate or triethylene glycol dimethacrylate, or combinations thereof. Preferably, the bifunctionalized acrylic monomeric units comprise EGDMA monomeric units. Even more preferably, at least 35%, preferably 100%, by weight of the bifunctionalized acrylic monomeric units, which are preferably in the above stated amounts, are EGDMA monomeric units.

In a preferred embodiment, the above amounts of monofunctionalized and bifunctionalized acrylic monomeric units are combined, preferably by their level of preference, and each amount is chosen such that the combined amount is 100% by weight with respect to the total weight of acrylic monomeric units, i.e. the acrylic monomeric units consist of monofunctionalized and bifunctionalized acrylic monomeric units. This applies to any embodiment described herein referring to a combination of monofunctionalized and bifunctionalized acrylic monomeric units.

The acrylic monomeric units comprised in the polymer network comprised in the hydrogels of the present invention comprise anionic acrylic monomeric units. An anionic acrylic monomeric unit stems from an acrylic acid monomer, or an alkylacrylic acid monomer, or a monomer containing at least one acrylic group and at least one anionic group. More particularly, a polymer network comprising anionic acrylic monomeric units is the result of the polymerization of a monomer mixture comprising acrylic acid monomers, or alkylacrylic acid monomers, or monomers containing at least one acrylic group and at least one anionic group. An anionic group refers to a group with a pKa of 5 or lower, such as of between −2 and 5. Where more than one deprotonation is possible, the pKa refers to the first pKa. Examples of such groups are carboxylic acids, sulphonic acids or phosphonic acids, preferably carboxylic acids or sulphonic acids, and more preferably carboxylic acids. An anionic acrylic monomeric unit wherein the anionic group is a carboxylic acid or a sulphonic acid is herein referred to as a carboxylic or sulphonic acid anionic acrylic monomeric unit, respectively.

Preferably, the anionic acrylic monomeric units are selected from acrylic acid monomeric units or alkylacrylic acid monomeric units, or monomeric units containing at least one acrylic group and at least one anionic group wherein the anionic group is connected to the non-carbonyl ester oxygen, or the amide nitrogen, of the acrylic group, through an alkyl linker, wherein the term alkyl has the meaning defined above. More preferably, the anionic acrylic monomeric units are selected from acrylic acid monomeric units or alkylacrylic acid monomeric units, even more preferably they are alkylacrylic acid monomeric units.

Non-limiting examples of anionic acrylic monomeric units are acrylic acid, methacrylic acid (MAA), 2-acrylamido-2-methylpropane sulfonic acid (AMPSA), methacryloyl-L-lysine, 3-sulfopropyl methacrylate, beta-carboxyethyl acrylate, 2-sulfoethyl methacrylate and 3-sulfopropyldimethyl-3-methacrylamidopropylammonium. Most preferably, the anionic acrylic monomeric units are selected from MAA or AMPSA monomeric units, and even more preferably, the anionic acrylic monomeric units are MAA monomeric units.

In a preferred embodiment, the acrylic monomeric units comprised in the polymer network comprised in the hydrogels of the present invention comprise an amount of between 0.001% and 5% by weight of anionic acrylic monomeric units with respect to the total weight of acrylic monomeric units; more preferably said amount is of between 0.002% and 2% by weight of anionic acrylic monomeric units with respect to the total weight of acrylic monomeric units. In an embodiment, the anionic acrylic monomeric units are carboxylic acid anionic acrylic monomeric units, and are present in an amount of between 0.1% and 5% by weight with respect to the total weight of acrylic monomeric units; more preferably said amount is of between 0.5% and 2% by weight with respect to the total weight of acrylic monomeric units. In a more particular embodiment, these amounts apply to the preferred anionic acrylic monomeric units, such as to MAA monomeric units.

In an embodiment, the anionic acrylic monomeric units are sulphonic acid anionic acrylic monomeric units, and are present in an amount of between 0.0010% and 0.0050% by weight with respect to the total weight of acrylic monomeric units; more preferably said amount is of between 0.0020% and 0.0041% by weight with respect to the total weight of acrylic monomeric units.

It should be noted that an anionic, aromatic or silicon oxide acrylic monomeric unit is necessarily also mono- or bi-functionalized, and therefore, the stated amount of the anionic, aromatic or silicon oxide acrylic monomeric unit is not in addition to the stated amount of mono- and bi-functionalized acrylic monomeric unit, but comprised in it. This applies throughout the present disclosure. In a preferred embodiment, the anionic, aromatic or silicon oxide acrylic monomeric unit is a monofunctionalized acrylic monomeric unit. Also, when ranges of amounts are provided for more than one of anionic, aromatic or silicon oxide acrylic monomeric units, amounts of each of these monomeric unit are chosen so as to never exceed 100% wt with respect to the total weight of acrylic monomeric units.

In an embodiment, the acrylic monomeric units comprised in the polymer network comprised in the hydrogels of the present invention comprise aromatic acrylic monomeric units. An aromatic acrylic monomeric unit stems from a monomer containing at least one acrylic group and at least one aromatic group. More particularly, a polymer network comprising aromatic acrylic monomeric units is the result of the polymerization of a monomer mixture comprising monomers containing at least one acrylic group and at least one aromatic group. An aromatic group refers to a group having a cyclic, planar, conjugated pi-electron system and may be a ring system comprising 2 aromatic rings, wherein the rings may be isolated, bridged or fused.

Preferably, the aromatic group comprises from 5 to 10 ring carbon atoms, such as phenyl or naphthyl, and may be substituted or unsubstituted. Preferably, in any embodiment described herein, the aromatic group comprises no nitrogen (N) heteroatoms. More preferably, in any embodiment described herein, the aromatic group comprises no nitrogen (N), oxygen (O) or sulfur(S) heteroatoms. Even more preferably, in any embodiment described herein, the aromatic group comprises no heteroatoms. Still more preferably, the aromatic group is a phenyl group, which may be substituted or unsubstituted. Substitution preferably refers to fluorination or perfluorination.

Preferably, the aromatic group is connected to the non-carbonyl ester oxygen, or the amide nitrogen, of the acrylic group, either directly; through an alkyl linker, wherein the term “alkyl” has the meaning defined above; or through an alkoxy linker, wherein the term “alkoxy” refers to an —O-alkyl group, wherein “alkyl” has the meaning defined above and wherein preferably the oxygen of the —O-alkyl group is directly connected to the aromatic group. Preferably, the aromatic group is connected through an alkyl linker.

Non-limiting examples of aromatic acrylic monomeric units are benzyl methacrylate (BzMA), ethylene glycol phenyl ether methacrylate (EGPEM), pentafluorophenyl acrylate, N-benzylmethacrylamide, phenyl acrylate, phenyl methacrylate, 2-phenylethyl acrylate, 2-phenylethyl methacrylate, benzyl acrylate and 2-hydroxy-3-phenoxypropyl methacrylate. Most preferably, the aromatic acrylic monomeric units are BzMA monomeric units.

In a preferred embodiment, the acrylic monomeric units comprised in the polymer network comprised in the hydrogels of the present invention comprise an amount of between 2% and 15% by weight of aromatic acrylic monomeric units with respect to the total weight of acrylic monomeric units; more preferably said amount is of between 5% and 8% by weight of aromatic acrylic monomeric units with respect to the total weight of acrylic monomeric units. In another embodiment, the same amounts apply to the preferred aromatic acrylic monomeric units, in particular BzMA monomeric units.

In a preferred embodiment, the acrylic monomeric units comprised in the polymer network comprised in the hydrogels of the present invention comprise:
an amount of at least 90% by weight of monofunctionalized acrylic monomeric units, preferably in any preferred form of these as described above, more preferably out of which at least 85% wt are HEMA monomeric units; and an amount of between 0.01% and 10% by weight of bifunctionalized acrylic monomeric units, preferably in any preferred form of these as described above, more preferably EGDMA monomeric units; with respect to the total weight of acrylic monomeric units;
an amount of between 0.0010% and 5% by weight of anionic acrylic monomeric units, preferably an amount of between 0.1% and 5% by weight of carboxylic acid anionic acrylic monomeric units, more preferably in any preferred form of these as described above, even more preferably MAA monomeric units, with respect to the total weight of acrylic monomeric units;
an amount of between 2% and 15% by weight of aromatic acrylic monomeric units, preferably in any preferred form of these as described above, preferably BzMA monomeric units, with respect to the total weight of acrylic monomeric units.

In further embodiments, preferred amounts for one, two, three or four of the four monomeric units are selected from those described above independently for each of the monomeric units.

In a more particular embodiment, in the above embodiment:
the monofunctionalized acrylic monomeric units consist of the MAA monomeric units, the BzMA monomeric units, and HEMA monomeric units;
the bifunctionalized acrylic monomeric units are EGDMA monomeric units;
the carboxylic acid anionic acrylic monomeric units are the MAA monomeric units; and
the aromatic monomeric units are the BzMA monomeric units.

In a preferred embodiment, the acrylic monomeric units comprised in the polymer network comprised in the hydrogels of the present invention comprise silicon oxide acrylic monomeric units. A silicon oxide acrylic monomeric unit stems from a monomer containing at least one acrylic group and at least one silicon oxide group. More particularly, a polymer network comprising silicon oxide acrylic monomeric units is the result of the polymerization of a monomer mixture comprising monomers containing at least one acrylic group and at least one silicon oxide group. A silicon oxide group refers to a group comprising a Si—O linkage.

Preferably, a Si atom in the silicon oxide group is connected to the non-carbonyl ester oxygen, or the amide nitrogen, of the acrylic group through an alkyl linker, wherein the term “alkyl” has the meaning defined above.

Alternatively or additionally, in a preferred embodiment, the silicon oxide group comprises at least one, such as up to six, preferably up to three, terminal —(OSiR′₃) groups, wherein each R′ is independently or simultaneously H or an alkyl group as described above, and preferably an alkyl group as described above. A preferred example of such monomeric unit is the 3-[Tris(trimethylsiloxy)silyl]propyl methacrylate (aka TRIS) unit.

Alternatively or additionally, in another preferred embodiment, the silicon oxide group comprises a non-terminal group of formula

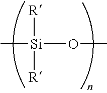

wherein each R′ is independently or simultaneously H or an alkyl group as described above, preferably an alkyl group as described above; and n is an integer number from 1 to 30; more preferably 4 to 20 (aka silicone). Particularly preferred are silicone acrylic monomeric units resulting from polymerization of acrylic monomers of the following formulae:

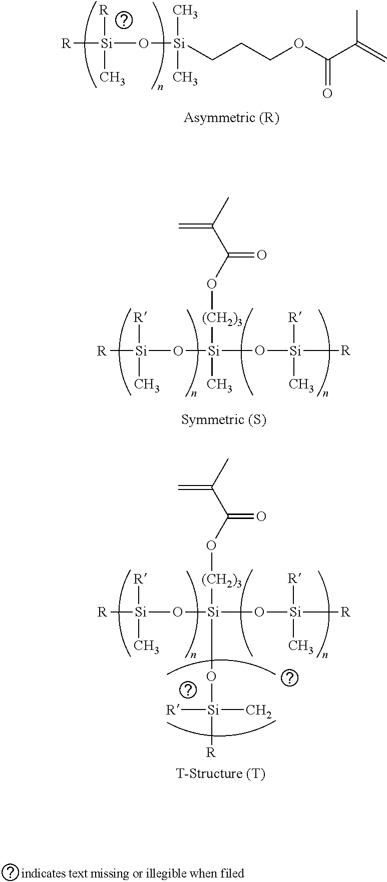

wherein each R′ and n independently have the above indicated meaning, and each R independently or simultaneously is methyl, n-butyl, HO(CH₂)₃— or CH₃O(CH₂)₃—. Non-limiting examples of silicon oxide acrylic monomeric units are monomethacryloxypropyl-sym-polydimethylsiloxane hydroxypropyl terminated (MCS-MC12), MCR-M11, MFR-M15, MCR-ME11, MCS-M11, MFS-M15, MCS-ME11, MCT-M11 (acronyms as described in Goff et al., Living Polymerization Routes to Siloxane Macromers and Higher Order Silicone Structures, Progress in Silicones and Silicone-Modified Materials ed S. Clarson 2013, Chapter 5, 59-78), (3-methacryloxy-2-hydroxypropoxy)propylbis(trimethylsiloxy)methylsilane, 3-acrylamidopropyltrimethoxysilane, 3-acrylamidopropyltris(trimethylsiloxy)silane, (2-acryloxyethoxy)trimethylsilane, n-(3-acryloxy-2-hydroxypropyl)-3-aminopropyltriethoxysilane, acryloxymethyltrimethoxysilane, (acryloxymethyl)phenethyltrimethoxysilane, acryloxymethyltrimethylsilane, (3-acryloxypropyl)methylbis(trimethylsiloxy)silane, (3-acryloxypropyl)methyldimethoxysilane, (3-acryloxypropyl)tris(trimethylsiloxy)silane, methacryloxypropyltrimethoxysilane, methacryloxypropyl terminated polydimethylsiloxane. Most preferably, the silicon oxide acrylic monomeric units are MCS-MC12, MCR-M11, MFR-M15, MCR-ME11, MCS-M11, MFS-M15, MCS-ME11 or MCT-M11 units, yet more preferably they are MCS-MC12 units.

In a preferred embodiment, the acrylic monomeric units comprised in the polymer network comprised in the hydrogels of the present invention comprise an amount of between 2% and 65% by weight of silicon oxide acrylic monomeric units with respect to the total weight of acrylic monomeric units; preferably said amount is of between 3% and 50%, more preferably of between 4 and 15%, even more preferably of between 5% and 8% by weight of silicon oxide acrylic monomeric units with respect to the total weight of acrylic monomeric units. In another embodiment, the same amounts apply to the preferred silicon oxide acrylic monomeric units, and more particularly to the MCS-MC12, MCR-M11, MFR-M15, MCR-ME11, MCS-M11, MFS-M15, MCS-ME11 or MCT-M11 units; even more particularly to the MCS-MC12 units.

In a preferred embodiment, the acrylic monomeric units comprised in the polymer network comprised in the hydrogels of the present invention comprise:
an amount of at least 90% by weight of monofunctionalized acrylic monomeric units, preferably in any preferred form of these as described above, more preferably out of which at least 85% wt are HEMA monomeric units; and an amount of between 0.01% and 10% by weight of bifunctionalized acrylic monomeric units, preferably in any preferred form of these as described above, more preferably EGDMA monomeric units; with respect to the total weight of acrylic monomeric units;
an amount of between 0.0010% and 5% by weight of anionic acrylic monomeric units, preferably an amount of between 0.1% and 5% by weight of carboxylic acid anionic acrylic monomeric units, more preferably in any preferred form of these as described above, even more preferably MAA monomeric units, with respect to the total weight of acrylic monomeric units;
an amount of between 2% and 15% by weight of silicon oxide acrylic monomeric units, preferably in any preferred form of these as described above, preferably MCS-MC12 monomeric units, with respect to the total weight of acrylic monomeric units.

In further embodiments, preferred amounts for one, two, three or four of the four monomeric units are selected from those described above independently for each of the monomeric units.

In a more particular embodiment, in the above embodiment:
the monofunctionalized acrylic monomeric units consist of the MAA monomeric units, the MCS-MC12 monomeric units and HEMA monomeric units;
the bifunctionalized acrylic monomeric units are EGDMA monomeric units;
the carboxylic acid anionic acrylic monomeric units are the MAA monomeric units; and
the silicon oxide acrylic monomeric units are the MCS-MC12 monomeric units.

In a very preferred embodiment, the acrylic monomeric units comprised in the polymer network comprised in the hydrogels of the present invention comprise silicon oxide acrylic monomeric units and aromatic acrylic monomeric units, more preferably in preferred forms of these as described above.

In a preferred embodiment, the acrylic monomeric units comprised in the polymer network comprised in the hydrogels of the present invention comprise:
an amount of at least 90% by weight of monofunctionalized acrylic monomeric units, preferably in any preferred form of these as described above, more preferably out of which at least 85% wt are HEMA monomeric units; and an amount of between 0.01% and 10% by weight of bifunctionalized acrylic monomeric units, preferably in any preferred form of these as described above, more preferably EGDMA monomeric units; with respect to the total weight of acrylic monomeric units;
an amount of between 0.0010% and 5% by weight of anionic acrylic monomeric units, preferably an amount of between 0.1% and 5% by weight of carboxylic acid anionic acrylic monomeric units, more preferably in any preferred form of these as described above, even more preferably MAA monomeric units, with respect to the total weight of acrylic monomeric units;
an amount of between 2% and 15% by weight of aromatic acrylic monomeric units, preferably in any preferred form of these as described above, preferably BzMA monomeric units, with respect to the total weight of acrylic monomeric units;
an amount of between 2% and 15% by weight of silicon oxide acrylic monomeric units, preferably in any preferred form of these as described above, preferably MCS-MC12 monomeric units, with respect to the total weight of acrylic monomeric units.

In further embodiments, preferred amounts for one, two, three, four or five of the five monomeric units are selected from those described above independently for each of the monomeric units.

In a more particular embodiment, in the above embodiment:
the monofunctionalized acrylic monomeric units consist of the MAA monomeric units, the BzMA monomeric units, the MCS-MC12 monomeric units and HEMA monomeric units;
the bifunctionalized acrylic monomeric units are EGDMA monomeric units;
the carboxylic acid anionic acrylic monomeric units are the MAA monomeric units;
the aromatic acrylic monomeric units are the BzMA monomeric units; and
the silicon oxide acrylic monomeric units are the MCS-MC12 monomeric units.

The following disclaimers apply to any of the hydrogel embodiments described herein.

In an embodiment, the hydrogel of the invention does not comprise a further polymer in addition to the polymeric network comprised in said hydrogel.

In an embodiment, the hydrogel of the invention or the polymer network comprised in the hydrogel of the present invention does not comprise molecular imprinting sites for atropine or a salt thereof.

As used herein, the term “molecular imprinting site” refers to a cavity in the polymer network of a hydrogel capable of selectively binding to a template molecule, e. g., atropine, typically present during polymerisation of the polymer network.

In an embodiment, the hydrogel of the invention or the polymer network comprised in the hydrogel of the present invention does not comprise vinylic monomeric units, preferably vinylpyrrolidone, vinylalcohol or propyleneimine monomeric units, more preferably vinylpyrrolidone monomeric units. Alternatively, the hydrogel of the invention or the polymer network comprised in the hydrogel of the present invention comprises these monomeric units in an amount of at most 1%, preferably at most 0.1%, by weight with respect to the total weight of the hydrogel or polymer network, respectively.

In an embodiment, the hydrogel of the invention or the polymer network comprised in the hydrogel of the present invention does not comprise amine monomeric units, more preferably non-acrylic amine monomeric units. Alternatively, the hydrogel of the invention or the polymer network comprised in the hydrogel of the present invention comprises these monomeric units in an amount of at most 1%, preferably at most 0.1%, by weight with respect to the total weight of the hydrogel or polymer network, respectively.

In an embodiment, the hydrogel of the invention or the polymer network comprised in the hydrogel of the present invention does not comprise alkylene oxide monomeric units, preferably ethylene oxide or propylene oxide monomeric units. Alternatively, the hydrogel of the invention or the polymer network comprised in the hydrogel of the present invention comprises these monomeric units in an amount of at most 1%, preferably at most 0.1%, by weight with respect to the total weight of the hydrogel or polymer network, respectively.

In an embodiment, the hydrogel of the invention or the polymer network comprised in the hydrogel of the present invention does not comprise saccharide monomeric units. Alternatively, the hydrogel of the invention or the polymer network comprised in the hydrogel of the present invention comprises these monomeric units in an amount of at most 1%, preferably at most 0.1%, by weight with respect to the total weight of the hydrogel or polymer network, respectively.

In an embodiment, the hydrogel of the invention or the polymer network comprised in the hydrogel of the present invention does not comprise non-acrylic Si-containing monomeric units, preferably non-acrylic siloxane monomeric units, more preferably non-acrylic dimethylsiloxane monomeric units. A non-acrylic Si-containing monomeric unit refers to a monomeric unit which is connected to the polymer backbone not through an acrylic group. Alternatively, the hydrogel of the invention or the polymer network comprised in the hydrogel of the present invention comprises these monomeric units in an amount of at most 1%, preferably at most 0.1%, by weight with respect to the total weight of the hydrogel or polymer network, respectively.

In an embodiment, the hydrogel of the invention or the polymer network comprised in the hydrogel of the present invention does not comprise non-acrylic anionic monomeric units. Alternatively, the hydrogel of the invention or the polymer network comprised in the hydrogel of the present invention comprises these monomeric units in an amount of at most 1%, preferably at most 0.1%, by weight with respect to the total weight of the hydrogel or polymer network, respectively.

In an embodiment, the hydrogel of the invention or the polymer network comprised in the hydrogel of the present invention does not comprise a UV light-blocking agent, preferably 2-(2′-hydroxy-5′-methacryloxyethylphenyl)-2H-benzotriazole or 2-(4-benzoyl-3-hydroxyphenoxy)ethyl acrylate. In an embodiment, the hydrogel of the invention or the polymer network comprised in the hydrogel of the present invention does not comprise a blue light-blocking agent, preferably 4-(phenyldiazenyl) phenyl methacrylate. Alternatively, the hydrogel of the invention or the polymer network comprised in the hydrogel of the present invention comprises these agents in an amount of at most 1%, preferably at most 0.1%, by weight with respect to the total weight of the hydrogel or polymer network, respectively.

In a particular embodiment, the hydrogel of the invention or the polymer network comprised in the hydrogel of the present invention does not comprise a photo-polymerization initiator, preferably 2-hydroxy-2-methylpropiophenone. Alternatively, the hydrogel of the invention or the polymer network comprised in the hydrogel of the present invention comprises photo-polymerization initiator in an amount of at most 1%, preferably at most 0.1%, by weight with respect to the total weight of the hydrogel or polymer network, respectively.

The polymer network comprised in the hydrogels of the invention is cross-linked. Cross-linking may be achieved by different manners known to the skilled person, such as by chemical means, such as by the inclusion of bifunctionalized acrylic monomers in the polymerization reaction.

In an embodiment, cross-linking is not achieved by freeze-thawing.

The hydrogel of the present invention comprises atropine.

The term “atropine” as used herein refers to atropine base or a salt thereof.

In a particular embodiment, the atropine comprised in the hydrogel of the present invention is in the form of atropine base.

In a preferred embodiment, the atropine comprised in the hydrogel is in the form of a salt of atropine, more preferably in the form of a salt comprising a multianion (i.e. an anion carrying two or more negative charges) and atropine cations (one cation per negative charge of the anion), even more preferably in the form of a salt comprising a di-anion and two atropine cations, such as atropine sulfate; or a hydrate thereof. More preferably, the salt of atropine is atropine sulfate monohydrate.

In a particular embodiment, the atropine comprised in the hydrogel of the invention is homogeneously distributed across the surface of the hydrogel of the present invention. In a particular embodiment, the atropine comprised in the hydrogel of the present invention is distributed forming a concentration gradient wherein the maximum atropine concentration occurs at the hydrogel surface and progressively decreases as the distance from any surface of the hydrogel increases.

In an embodiment, the atropine is comprised in the hydrogel of the invention in an amount of at least 0.01%; preferably at least 0.05%; more preferably at least 0.1%; by weight with respect to the total weight of the hydrogel. Alternatively, the atropine is comprised in the hydrogel of the invention in an amount of at least 0.1; preferably at least 0.5; more preferably at least 1; mg per g of hydrogel.

In a particular embodiment, the atropine is comprised in the hydrogel of the invention in any of the above amounts up to an amount of 2%, preferably up to 1%, even more preferably up to 0.5% by weight with respect to the total weight of the hydrogel, such as in an amount of 0.01% to 2%; preferably 0.05% to 1%; more preferably 0.1% to 0.5%; by weight with respect to the total weight of the hydrogel. Alternatively, the atropine is comprised in the hydrogel of the invention in any of the above amounts up to an amount of 20, preferably up to 10, even more preferably up to 5; mg per g of hydrogel, such as in an amount of 0.1 to 20; preferably 0.5 to 10; more preferably 1 to 5; mg per g of hydrogel.

The above weights are based on the dry weight of the hydrogel, i.e. a hydrogel comprising no water.

In a particular embodiment, the same amounts apply to atropine salts as described above, particularly to atropine sulfate, more particularly to atropine sulfate monohydrate.

In a particular and preferred embodiment, atropine, preferably atropine sulfate, is comprised in the hydrogel in an amount of 0.5 to 10 mg per g of hydrogel, and the acrylic monomeric units comprised in the polymer network comprised in the hydrogels of the present invention comprise:
an amount of at least 90% by weight of monofunctionalized acrylic monomeric units, preferably in any preferred form of these as described above, more preferably out of which at least 85% wt are HEMA monomeric units; and an amount of between 0.01% and 10% by weight of bifunctionalized acrylic monomeric units, preferably in any preferred form of these as described above, more preferably EGDMA monomeric units; with respect to the total weight of acrylic monomeric units;
an amount of between 0.0010% and 5% by weight of anionic acrylic monomeric units, preferably an amount of between 0.1% and 5% by weight of carboxylic acid anionic acrylic monomeric units, more preferably in any preferred form of these as described above, even more preferably MAA monomeric units, with respect to the total weight of acrylic monomeric units;
an amount of between 2% and 15% by weight of aromatic acrylic monomeric units, preferably in any preferred form of these as described above, preferably BzMA monomeric units, with respect to the total weight of acrylic monomeric units;
an amount of between 2% and 15% by weight of silicon oxide acrylic monomeric units, preferably in any preferred form of these as described above, preferably MCS-MC12 monomeric units, with respect to the total weight of acrylic monomeric units.

It has unexpectedly been found that when the hydrogels of the present invention comprise acrylic monomeric units comprising anionic groups, combined with acrylic monomeric units comprising aromatic and/or acrylic monomeric units comprising siloxane oxide groups; then the hydrogels are capable of loading high quantities of atropine and the hydrogels also exhibit sustained release of said atropine over extended periods.

The term “sustained release” is used in a conventional sense relating to a gradual release of a compound during a period of time and preferably, although not necessarily, with relatively constant compound release levels over a long period of time.

Alternatively or additionally, the hydrogels of the present invention can be characterised by their atropine release rate when the release of atropine is measured in NaCl 0.9% solution in an incubating shaker at about 36° C. and a shaking rotation speed of 180 rpm, more specifically according to Example 3 hereinbelow.

In an embodiment, for the same aforementioned experimental conditions, the atropine comprised in the hydrogel of the present invention, is released in less than 100% by weight, relative to the total atropine released at 24 h, within 1 hour (i. e., within the first hour of release).

In a particular embodiment, the atropine comprised in the hydrogel of the present invention is released in an amount of less than 99% by weight, relative to the total atropine released at 24 h, within 1 hour. In a more particular embodiment, the atropine comprised in the hydrogel of the present invention is released in an amount of less than 95%, by weight, relative to the total atropine released at 24 h, within 1 hour.

In an embodiment, for the same aforementioned experimental conditions, the atropine comprised in the hydrogel of the present invention is released in an amount of 70% to less than 100%, preferably 70% to 99%, more preferably 70% to 95% by weight, relative to the total atropine released at 24 h, within 1 hour. In an embodiment, for the same aforementioned experimental conditions, the atropine comprised in the hydrogel of the present invention is released in an amount of less than 90% preferably less than 85%, more preferably less than 80% by weight, relative to the total atropine released at 24 h, within 1 hour; and the acrylic monomeric units comprised in the polymer network comprised in the hydrogels of the present invention comprise:
an amount of at least 90% by weight of monofunctionalized acrylic monomeric units, preferably in any preferred form of these as described above, more preferably out of which at least 85% wt are HEMA monomeric units; and an amount of between 0.01% and 10% by weight of bifunctionalized acrylic monomeric units, preferably in any preferred form of these as described above, more preferably EGDMA monomeric units; with respect to the total weight of acrylic monomeric units;
an amount of between 0.0010% and 5% by weight of anionic acrylic monomeric units, preferably an amount of between 0.1% and 5% by weight of carboxylic acid anionic acrylic monomeric units, more preferably in any preferred form of these as described above, even more preferably MAA monomeric units, with respect to the total weight of acrylic monomeric units;
an amount of between 2% and 15% by weight of aromatic acrylic monomeric units, preferably in any preferred form of these as described above, preferably BzMA monomeric units, with respect to the total weight of acrylic monomeric units;
an amount of between 2% and 15% by weight of silicon oxide acrylic monomeric units, preferably in any preferred form of these as described above, preferably MCS-MC12 monomeric units, with respect to the total weight of acrylic monomeric units.

In an embodiment, for the same aforementioned experimental conditions, the atropine comprised in the hydrogel of the present invention is released in an amount of 60% to 90%, preferably 60% to 85%, more preferably 60% to 80% by weight, relative to the total atropine released at 24 h, within 1 hour; and the acrylic monomeric units comprised in the polymer network comprised in the hydrogels of the present invention comprise:
an amount of at least 90% by weight of monofunctionalized acrylic monomeric units, preferably in any preferred form of these as described above, more preferably out of which at least 85% wt are HEMA monomeric units; and an amount of between 0.01% and 10% by weight of bifunctionalized acrylic monomeric units, preferably in any preferred form of these as described above, more preferably EGDMA monomeric units; with respect to the total weight of acrylic monomeric units;
an amount of between 0.0010% and 5% by weight of anionic acrylic monomeric units, preferably an amount of between 0.1% and 5% by weight of carboxylic acid anionic acrylic monomeric units, more preferably in any preferred form of these as described above, even more preferably MAA monomeric units, with respect to the total weight of acrylic monomeric units;
an amount of between 2% and 15% by weight of aromatic acrylic monomeric units, preferably in any preferred form of these as described above, preferably BzMA monomeric units, with respect to the total weight of acrylic monomeric units;
an amount of between 2% and 15% by weight of silicon oxide acrylic monomeric units, preferably in any preferred form of these as described above, preferably MCS-MC12 monomeric units, with respect to the total weight of acrylic monomeric units.

The second aspect of the invention relates to a precursor hydrogel comprising:
a polymer network, wherein the polymer network:
   comprises acrylic monomeric units in an amount of at least 90% by weight with respect to the total weight of the polymer network; and
   is cross-linked;
wherein the acrylic monomeric units comprised in the polymer network comprise anionic acrylic monomeric units; and
wherein the acrylic monomeric units comprised in the polymer network comprise aromatic acrylic monomeric units and/or comprise silicon oxide acrylic monomeric units, preferably wherein the aromatic groups comprised in said aromatic acrylic monomeric units comprise no N heteroatoms, more preferably comprise no N, O or S heteroatoms, even more preferably comprise no heteroatoms.

As used herein, “precursor hydrogel” refers to the hydrogel which does not comprise atropine. One skilled in the art will understand that all the preferred and particular embodiments described above for the polymer network comprised in the hydrogel of the present invention will be applicable for the precursor hydrogel described in this section.

In a preferred embodiment, the acrylic monomeric units comprised in the polymer network comprised in the precursor hydrogel of the present invention comprise:
an amount of at least 90% by weight of monofunctionalized acrylic monomeric units, preferably in any preferred form of these as described above, more preferably out of which at least 85% wt are HEMA monomeric units; and an amount of between 0.01% and 10% by weight of bifunctionalized acrylic monomeric units, preferably in any preferred form of these as described above, more preferably EGDMA monomeric units; with respect to the total weight of acrylic monomeric units;
an amount of between 0.0010% and 5% by weight of anionic acrylic monomeric units, preferably an amount of between 0.1% and 5% by weight of carboxylic acid anionic acrylic monomeric units, more preferably in any preferred form of these as described above, even more preferably MAA monomeric units, with respect to the total weight of acrylic monomeric units;
an amount of between 2% and 15% by weight of aromatic acrylic monomeric units, preferably in any preferred form of these as described above, preferably BzMA monomeric units, with respect to the total weight of acrylic monomeric units.

In further embodiments, preferred amounts for one, two, three or four of the four monomeric units are selected from those described above independently for each of the monomeric units.

In a more particular embodiment, in the above embodiment:
the monofunctionalized acrylic monomeric units consist of the MAA monomeric units, the BzMA monomeric units, and HEMA monomeric units;
the bifunctionalized acrylic monomeric units are EGDMA monomeric units;
the carboxylic acid anionic acrylic monomeric units are the MAA monomeric units; and
the aromatic acrylic monomeric units are the BzMA monomeric units.

In a preferred embodiment, the acrylic monomeric units comprised in the polymer network comprised in the precursor hydrogel of the present invention comprise:
an amount of at least 90% by weight of monofunctionalized acrylic monomeric units, preferably in any preferred form of these as described above, more preferably out of which at least 85% wt are HEMA monomeric units; and an amount of between 0.01% and 10% by weight of bifunctionalized acrylic monomeric units, preferably in any preferred form of these as described above, more preferably EGDMA monomeric units; with respect to the total weight of acrylic monomeric units;
an amount of between 0.0010% and 5% by weight of anionic acrylic monomeric units, preferably an amount of between 0.1% and 5% by weight of carboxylic acid anionic acrylic monomeric units, more preferably in any preferred form of these as described above, even more preferably MAA monomeric units, with respect to the total weight of acrylic monomeric units;
an amount of between 2% and 15% by weight of silicon oxide acrylic monomeric units, preferably in any preferred form of these as described above, preferably MCS-MC12 monomeric units, with respect to the total weight of acrylic monomeric units.

In further embodiments, preferred amounts for one, two, three or four of the four monomeric units are selected from those described above independently for each of the monomeric units.

In a more particular embodiment, in the above embodiment:
the monofunctionalized acrylic monomeric units consist of the MAA monomeric units, the MCS-MC12 monomeric units and HEMA monomeric units;
the bifunctionalized acrylic monomeric units are EGDMA monomeric units;
the carboxylic acid anionic acrylic monomeric units are the MAA monomeric units; and
the silicon oxide acrylic monomeric units are the MCS-MC12 monomeric units.

In a preferred embodiment, the acrylic monomeric units comprised in the polymer network comprised in the precursor hydrogel of the present invention comprise:
an amount of at least 90% by weight of monofunctionalized acrylic monomeric units, preferably in any preferred form of these as described above, more preferably out of which at least 85% wt are HEMA monomeric units; and an amount of between 0.01% and 10% by weight of bifunctionalized acrylic monomeric units, preferably in any preferred form of these as described above, more preferably EGDMA monomeric units; with respect to the total weight of acrylic monomeric units;
an amount of between 0.0010% and 5% by weight of anionic acrylic monomeric units, preferably an amount of between 0.1% and 5% by weight of carboxylic acid anionic acrylic monomeric units, more preferably in any preferred form of these as described above, even more preferably MAA monomeric units, with respect to the total weight of acrylic monomeric units;
an amount of between 2% and 15% by weight of aromatic acrylic monomeric units, preferably in any preferred form of these as described above, preferably BzMA monomeric units, with respect to the total weight of acrylic monomeric units;
an amount of between 2% and 15% by weight of silicon oxide acrylic monomeric units, preferably in any preferred form of these as described above, preferably MCS-MC12 monomeric units, with respect to the total weight of acrylic monomeric units.

In further embodiments, preferred amounts for one, two, three, four or five of the five monomeric units are selected from those described above independently for each of the monomeric units.

In a more particular embodiment, in the above embodiment:
the monofunctionalized acrylic monomeric units consist of the MAA monomeric units, the BzMA monomeric units, the MCS-MC12 monomeric units and HEMA monomeric units;
the bifunctionalized acrylic monomeric units are EGDMA monomeric units;
the carboxylic acid anionic acrylic monomeric units are the MAA monomeric units;
the aromatic acrylic monomeric units are the BzMA monomeric units; and
the silicon oxide acrylic monomeric units are the MCS-MC12 monomeric units.

In an embodiment, in any embodiment described herein relating to the precursor hydrogel, the precursor hydrogel does not comprise Bimatoprost or any salt thereof, more preferably the precursor hydrogel does not comprise any active pharmaceutical ingredient, more preferably the precursor hydrogel consists of the polymer network and optionally solvent.

In an embodiment, in any embodiment described herein relating to the precursor hydrogel the precursor hydrogel or the polymer network comprised in the precursor hydrogel does not comprise molecular imprinting sites for atropine or a salt thereof.

The third aspect of the present invention relates to a method for preparing the hydrogels of the invention.

In an embodiment, the method of the invention comprises the steps of:
a) Providing a precursor hydrogel comprising a polymer network, wherein the polymer network:
   comprises acrylic monomeric units in an amount of at least 90% by weight with respect to the total weight of the polymer network, wherein said acrylic monomeric units comprise anionic acrylic monomeric units; and wherein said acrylic monomeric units comprise aromatic acrylic monomeric units and/or silicon oxide acrylic monomeric units, preferably wherein the aromatic groups comprised in said aromatic acrylic monomeric units comprise no N heteroatoms, more preferably comprise no N, O or S heteroatoms, even more preferably comprise no heteroatoms;
   is cross-linked;
b) Providing an atropine solution;
c) Combining the precursor hydrogel of step a) and the atropine solution of step b).

Step a) requires providing the precursor hydrogel. The precursor hydrogel of the present invention can be prepared by methods of monomer mixture polymerization well-known to the person skilled in the art. It is understood that the monomers in the mixture must be polymerizable, i.e. they must comprise a functional group which can react with other monomers in a polymerization reaction. For instance, polymer networks comprising acrylic monomeric units are produced from a mixture of acrylic monomers, i.e. monomers comprising at least one acrylic group, which comprises the polymerizable C═C double bond. Such monomers are readily commercially available, as described in the Examples.

Every embodiment provided herein describing the nature and amounts of the monomeric units comprised in the polymer network comprised in the hydrogels of the present invention applies mutatis mutandis to the precursor hydrogel. Similarly, the person skilled in the art knows how to translate the composition of said hydrogel into the corresponding non-polymerized monomer mixture.

In an embodiment, every embodiment provided herein describing the nature and amounts of the monomeric units comprised in the polymer network comprised in the hydrogels of the present invention refers rather to the nature and amount of the corresponding monomers in the non-polymerized monomer mixture that is to be subjected to polymerization. Thus, by way of example, when the acrylic monomeric units comprised in the polymer network comprised in the hydrogels of the present invention comprise an amount of between 2% and 15% by weight of silicon oxide acrylic monomeric units with respect to the total weight of acrylic monomeric units, this automatically generates an embodiment wherein the acrylic monomers comprised in the monomer mixture that is to be subjected to polymerization comprise an amount of between 2% and 15% by weight of silicon oxide acrylic monomers with respect to the total weight of acrylic monomers in said mixture. The monomer mixture refers to the group of compounds, such as monomers and initiator, that end up forming the polymer network.

Polymerization reactions include thermal- or photo-polymerization employing a radical initiator.

Examples of thermal polymerization initiator types are peroxides, hydroperoxides, azo-bis(alkyl- or cycloalkyl-nitriles), persulfates, percarbonates or mixtures thereof. Specific examples are benzoylperoxide, tert.-butyl peroxide, di-tert.-butyl-diperoxyphthalate, tert.-butyl hydroperoxide, azo-bis(isobutyronitrile) (AIBN), 1,1-azodiisobutyramidine, 1,1′-azo-bis(1-cyclohexanecarbonitrile), 2,2′-azo-bis(2,4-dimethylvaleronitrile). Preferably, the initiator is a thermal polymerization initiator, and more preferably it is AIBN.

The polymerization is carried out by subjecting the monomer mixture to an elevated temperature, for example to a temperature of from 30 to 100° C. and preferably 40 to 80° C. The reaction time may vary within wide limits, but is conveniently, for example, from 1 to 48 hours. In a preferred embodiment, the polymerization is carried out at 30-70° C. for 6 to 18 h and then at 50-90° C. for 18 to 30 h; such as 50° C. for 12 h and then at 70° C. for 30 h. It is advantageous to previously degas the components and solvents, if any, used in the polymerization reaction and to carry out said polymerization reaction under an inert atmosphere, for example under a nitrogen or argon atmosphere.

Examples of suitable solvents include, without limitation, tetrahydrofuran, tripropylene glycol methyl ether, dipropylene glycol methyl ether, ethylene glycol n-butyl ether, ketones (e.g., acetone, methyl ethyl ketone, etc.), diethylene glycol n-butyl ether, diethylene glycol methyl ether, ethylene glycol phenyl ether, propylene glycol methyl ether, propylene glycol methyl ether acetate, dipropylene glycol methyl ether acetate, propylene glycol n-propyl ether, dipropylene glycol n-propyl ether, tripropylene glycol n-butyl ether, propylene glycol n-butyl ether, dipropylene glycol n-butyl ether, tripropylene glycol n-butyl ether, propylene glycol phenyl ether dipropylene glycol dimetyl ether, polyethylene glycols, polypropylene glycols, ethyl acetate, butyl acetate, amyl acetate, methyl lactate, ethyl lactate, i-propyl lactate, methylene chloride, 2-butanol, 1-propanol, 2-propanol, menthol, cyclohexanol, cyclopentanol and exonorborneol, 2-pentanol, 3-pentanol, 2-hexanol, 3-hexanol, 3-methyl-2-butanol, 2-heptanol, 2-octanol, 2-nonanol, 2-decanol, 3-octanol, norborneol, tert-butanol, tert-amyl, alcohol, 2-methyl-2-pentanol, 2,3-dimethyl-2-butanol, 3-methyl-3-pentanol, 1-methylcyclohexanol, 2-methyl-2-hexanol, 3,7-dimethyl-3-octanol, 1-chloro-2-methyl-2-propanol, 2-methyl-2-heptanol, 2-methyl-2-octanol, 2-2-methyl-2-nonanol, 2-methyl-2-decanol, 3-methyl-3-hexanol, 3-methyl-3-heptanol, 4-methyl-4-heptanol, 3-methyl-3-octanol, 4-methyl-4-octanol, 3-methyl-3-nonanol, 4-methyl-4-nonanol, 3-methyl-3-octanol, 3-ethyl-3-hexanol, 3-methyl-3-heptanol, 4-ethyl-4-heptanol, 4-propyl-4-heptanol, 4-isopropyl-4-heptanol, 2,4-dimethyl-2-pentanol, 1-methylcyclopentanol, 1-ethylcyclopentanol, 1-ethylcyclopentanol, 3-hydroxy-3-methyl-1-butene, 4-hydroxy-4-methyl-1-cyclopentanol, 2-phenyl-2-propanol, 2-methoxy-2-methyl-2-propanol 2,3,4-trimethyl-3-pentanol, 3,7-dimethyl-3-octanol, 2-phenyl-2-butanol, 2-methyl-1-phenyl-2-propanol and 3-ethyl-3-pentanol, 1-ethoxy-2-propanol, 1-methyl-2-propanol, t-amyl alcohol, isopropanol, 1-methyl-2-pyrrolidone, N,N-dimethylpropionamide, dimethyl formamide, dimethyl acetamide, dimethyl propionamide, N-methyl pyrrolidinone, Dimethyl sulfoxide and mixtures thereof. When the monomer mixture comprises sulphonic acid anionic acrylic monomers, such as AMPSA, then the solvent preferably comprises dimethyl sulfoxide.

In a preferred embodiment, however, the polymerization is carried out in the absence of solvent.

Examples of photo-polymerization initiators (aka photoinitiators) are benzoin methyl ether, 2,4,6-trimethylbenzoyldiphenylophosphine oxide, bis-(2,6-dichlorobenzoyl)-4-N-propylphenylphosphine oxide, bis-(2,6-dichlorobenzoyl)-4-N-butylphenylphosphine oxide, diethoxyacetophenone, 1-hydroxycyclohexyl phenyl ketone or Germane-based Norrish Type I photoinitiators. The polymerization can be triggered off by exposing a mixture comprising the monomer mixture and the photoinitiator to actinic radiation, for example light, in particular UV light.

In a particular embodiment, however, the polymerization is carried out in the absence of a photoinitiator, preferably 2-hydroxy-2-methylpropiophenone.

Polymerization reactions may be carried out using molecular imprinting polymerization. This polymerization process usually involves carrying out the polymerization of monomers in the presence of a template molecule, e. g. atropine or a salt thereof, that is extracted after polymerization leaving behind cavities complementary to said template molecule, also referred to as molecular imprinting sites, in the polymer network. Complementarity can refer to steric complementarity or chemical complementarity, or both.

In a particularly preferred embodiment, however, the polymerization is not carried out using molecular imprinting, more particularly molecular imprinting with atropine as a template molecule, for the formation of molecular imprinted sites in the polymer network. In a preferred embodiment, the polymerization is carried out in the absence of atropine or a salt thereof, such that the precursor hydrogel of step a) does not comprise molecular imprinting sites for atropine or a salt thereof.

Most preferably, the hydrogel precursor is prepared by a method as described in Vivero-Lopez et al., Phosphorylcholine-Based Contact Lenses for Sustained Release of Resveratrol: Design, Antioxidant and Antimicrobial Performances, and In Vivo Behavior, ACS Appl. Mater. Interfaces 2022, 14 (50): 55431-55446.

In an embodiment, after the polymerization and prior to subjecting the precursor hydrogel to step c), unreacted monomers are removed from the precursor hydrogel, such as by washing it with a solvent, such as an aqueous solvent, e.g. water and/or an aqueous saline solution such as a NaCl solution at a concentration of 0.9%, until no monomer is detected in the solvent that was used for washing. Alternatively, unreacted monomers are removed from the precursor hydrogel by subjecting the precursor hydrogel to boiling, such as by immersing the precursor hydrogel in boiling water.

Additionally or alternatively, after the polymerization and prior to subjecting the precursor hydrogel to step c), the precursor hydrogel is dried, such as by heating it above room temperature (25° C.). In an exemplary embodiment, when the hydrogel is in the form of a contact lens, the hydrogel can be dried at a temperature of 40° C. for 2 hours and then at 70° C. for a further 2 hours.

Step b) requires providing an atropine solution. The term atropine has the meaning that was provided hereinabove. Providing the atropine in said solution form facilitates the penetration of the atropine molecule throughout the polymer network comprised in the precursor hydrogel and thus maximises the efficiency of step c) in the method of the present invention. In a preferred embodiment, the atropine salt in the atropine solution of step b) is atropine sulfate, more particularly atropine sulfate monohydrate.

Examples of suitable solvents for the atropine solution of step b) include, without limitation, water and organic solvents such as alkanols (e.g. methanol, ethanol), THF, DMSO, dimethyl formamide or mixtures thereof. The atropine solution may comprise any of these solvents or a mixture thereof, preferably a mixture wherein the water is predominant such as a mixture wherein the volumetric ratio of water to organic solvent is 5:1 or greater, preferably 10:1 or greater. More preferably the atropine solution is an aqueous solution, such as a water solution.

In a particular embodiment, the atropine solution is a buffered saline solution.

In a preferred embodiment, the solution is an aqueous saline solution such as a NaCl solution, such as a NaCl solution at a concentration of 0.9% (0.154 M).

In another particular embodiment, the solution is an isotonic solution. The term “isotonic” refers to a solution in which its effective osmole concentration is the same as the solute concentration of another solution or another physiological fluid with which it is compared.

In an embodiment, the solution is a desalinated solution, such as distilled water. The term “desalinated” herein implies that the concentration of salts, such as of NaCl, is of 0.015 M or lower.

In an embodiment, the concentration of atropine in the atropine solution of step b) is between 0.01 and 20 mg/mL, preferably between 0.05 and 20 mg/mL, more preferably between 0.1 and 20 mg/mL, even more preferably between 0.15 and 20 mg/mL, still more preferably between 0.2 and 20 mg/mL.

In an embodiment, the concentration of atropine in the atropine solution of step b) is between 0.01 and 10 mg/mL, preferably between 0.05 and 10 mg/mL, more preferably between 0.1 and 10 mg/mL, even more preferably between 0.15 and 10 mg/mL, still more preferably between 0.2 and 10 mg/mL.

In an embodiment, the concentration of atropine in the atropine solution of step b) is between 0.01 and 5 mg/mL, preferably between 0.05 and 5 mg/mL, more preferably between 0.1 and 5 mg/mL, even more preferably between 0.15 and 5 mg/mL, still more preferably between 0.2 and 5 mg/mL.

In an embodiment, the concentration of atropine in the atropine solution of step b) is between 0.01 and 1.5 mg/mL, preferably between 0.05 and 1.5 mg/mL, more preferably between 0.1 and 1.5 mg/mL, even more preferably between 0.15 and 1.5 mg/mL, still more preferably between 0.2 and 1.5 mg/mL.

In an embodiment, the concentration of atropine in the atropine solution of step b) is between 0.01 and 0.5 mg/mL, preferably between 0.05 and 0.5 mg/mL, more preferably between 0.1 and 0.5 mg/mL, even more preferably between 0.15 and 0.5 mg/mL, still more preferably between 0.2 and 0.5 mg/mL.

Step c) requires bringing said precursor into contact (combining) the precursor hydrogel with the atropine solution of step b).

Different methods known in the art can be employed for the combining step of the precursor hydrogel and the atropine solution, such as immersing the precursor hydrogel in the atropine solution, drop-coating the atropine solution onto the precursor hydrogel, spraying the atropine solution onto the precursor hydrogel, or applying the atropine solution by a brush onto the precursor hydrogel. Any method may be used as long as atropine solution can be absorbed by the precursor hydrogel. Preferably, the combining of the precursor hydrogel and the atropine solution is by immersing the precursor hydrogel in the atropine solution, more preferably in an atropine solution as described above, even more preferably in an aqueous atropine sulfate solution as described above.

The combining of the precursor hydrogel and the atropine solution is preferably performed for a period of time sufficient for the atropine solution to be absorbed throughout the precursor hydrogel. Preferably, combining or bringing into contact is by immersion as described above, and the precursor hydrogel is maintained immersed in the atropine solution for a period of at least 1 day, more preferably of at least 2 days, such as 2 days. The combining of the precursor hydrogel and the atropine solution may be performed at any temperature which neither evaporates nor freezes either component, but can generally and preferably be carried out at room temperature (15 to 25° C.).

In a preferred particular embodiment, the combining of the precursor hydrogel and the atropine solution is performed by immersing the precursor hydrogel in an aqueous atropine sulfate solution for a period of at least 2 days and at room temperature.

It has unexpectedly been found that the hydrogels of the present invention exhibit increased atropine loading capacity and are able to release said atropine in a sustained manner despite being prepared by combination of the already synthesised polymer network and atropine.

In an embodiment, the present invention relates to a hydrogel of the present invention in the form of a contact lens. Alternatively, in a further aspect, the present invention relates to a contact lens comprising a hydrogel according to the present invention. In another aspect, the present invention relates to a method for preparing said contact lens.

In an embodiment, the contact lens is a soft contact lens. In the context of the present invention, a soft contact lens is a contact lens having an elastic modulus (i.e., Young's modulus) of less than 2.5 MPa.

Different methods for preparing the contact lens may be employed. For instance, the polymerization reaction of step a) of the methods of the present invention can be carried out in a mold with a contact lens shape, such that the precursor hydrogel is already provided in the form of a contact lens. If the polymerization reaction has not been carried out in a mold providing the hydrogel with a contact lens shape, the contact lens can be prepared by means of lathe-cutting the precursor hydrogel or the hydrogel of the invention, or by means of molding the same, particularly by means of centrifugal molding or by means of cast molding, or by means of combinations of these techniques.

Further aspects of the invention relate to the hydrogels of the invention, preferably in the form of a contact lens, for use in medicine, preferably in ophthalmology. In a further aspect, the present invention relates to the hydrogels of the invention for use in the prevention and/or treatment of an ophthalmic condition, preferably for use in the prevention and/or treatment of myopia.

The present invention also embraces methods of prevention and/or treatment of an ophthalmic condition, preferably myopia, in a subject in need thereof, comprising administering a hydrogel according to the present invention to said subject.

The present invention also embraces the use of a hydrogel according to the present invention in the manufacture of a medicament for the prevention and/or treatment of an ophthalmic condition, preferably myopia.

Ophthalmic conditions that may be prevented or treated with atropine are myopia; uveitis; amblyopia; accommodative spasms; nerve agent or insecticide toxicity; adhesion of the iris to the anterior lens in cases of floppy iris syndrome; inflammatory processes (as neoadjuvant therapy). Atropine is also used as mydriatic and cycloplegic agent during eye examinations.

The hydrogels of the present invention can be formulated for use in human or veterinary medicine, preferably human medicine.

The prevention and/or treatment of an ophthalmic condition, preferably myopia, comprises placing the hydrogel, preferably in the form of a contact lens, of the invention, on the eye of the subject, more particularly on the cornea of the subject. Preferably, the contact lens remains on the eye of the subject for a period of at least 4 hours, at least 6 hours, or at least 8 hours; such as for any of these periods up to one day, or up to 12 hours; more preferably, for overnight use, this is, for use during the hours of night sleep.

Preferably, the prevention and/or treatment of an ophthalmic condition, preferably myopia, as described above comprises administering atropine to one or more, preferably to all, of the aqueous humor (AH), cornea, vitreous humor (VH), retina and sclera.

The atropine is administered in therapeutically effective amounts. The term “therapeutically effective amount” refers to an amount which, when administered to a living subject, achieves the desired therapeutic effect on the living subject. In general, the therapeutically effective amount will depend on the nature and severity of the disorder being treated. The exact amount will be ascertainable by one skilled in the medical art.

### EXAMPLES

### Materials

2-Hydroxyethyl methacrylate (HEMA) was from Merck KGaA (Darmstadt, Germany); benzyl methacrylate (BzMA) was from Polysciences Inc. (Warrington, UK); monomethacryloxypropyl-sym-polydimethylsiloxane hydroxypropyl terminated (MCS-MC12) was from Gelest, Inc. (Morrisville, USA); ethylene glycol dimethacrylate (EGDMA), methacrylic acid (MAA), 2,2′-azobis(2-methylpropionitrile) (AIBN) and sodium dodecyl sulfate were from Sigma-Aldrich (Steinheim, Germany). Atropine sulfate monohydrate (MW 694.83 g/mol) was purchased from Saurav Chemicals Limited (Punjab, India). Sodium chloride (NaCl) was from Labkem (Barcelona, Spain). Potassium di-hydrogen phosphate (KH₂PO₄) was from PanReac Química S.L.U. (Barcelona, Spain). Acetonitrile for HPLC LC-MS grade was from VWR Chemicals (Fontenary-sous-Bois, France). Ultrapure water (resistivity >18.2 MΩ·cm) was obtained by reverse osmosis (MilliQ®, Millipore Ibérica, Madrid, Spain). Schirmer test strips were from Contactcare Ophthalmics and Diagnostics (Gujarat, India).

### Example 1—Precursor Hydrogel Synthesis

Various precursor hydrogel batches differing in monomeric unit composition (see Table 1 for % weight composition and structures of monomers below Table 1) were prepared as previously described [Vivero-Lopez et al., Phosphorylcholine-Based Contact Lenses for Sustained Release of Resveratrol: Design, Antioxidant and Antimicrobial Performances, and In Vivo Behavior, ACS Appl. Mater. Interfaces 2022, 14 (50): 55431-55446]. Briefly, monomer solutions comprising AIBN as initiator were added (60 μL) into curved polypropylene molds typically used for preparing daily disposable CLs (n=20). The polymerization was carried out at 50° C. for 12 h and 70° C. for other 30 h. After polymerization, the CLs were demolded and alternatively washed under magnetic stirring in 500 mL of MilliQ® water and NaCl 0.9% solution until the complete removal of unreacted monomers. Finally, the CLs were dried at 40° C. for 2 h and 70° C. for other 2 h to be used in further experiments.

| | | | | | MCS- |
|---|---|---|---|---|---|
| ID | HEMA | MAA | EGDMA | BzMA | MC12 |
| B2x | 91.9460 | 0.8435 | 0.2962 | 6.9143 | 0.0000 |
| B3x | 91.1748 | 1.6752 | 0.2937 | 6.8563 | 0.0000 |
| S2b | 86.4949 | 0.8501 | 0.2985 | 0.0000 | 12.3564 |
| S3b | 85.7637 | 1.6883 | 0.2960 | 0.0000 | 12.2520 |
| S2x | 86.4949 | 0.8501 | 0.2985 | 6.1782 | 6.1782 |
| S3x | 85.7637 | 1.6883 | 0.2960 | 6.1260 | 6.1260 |

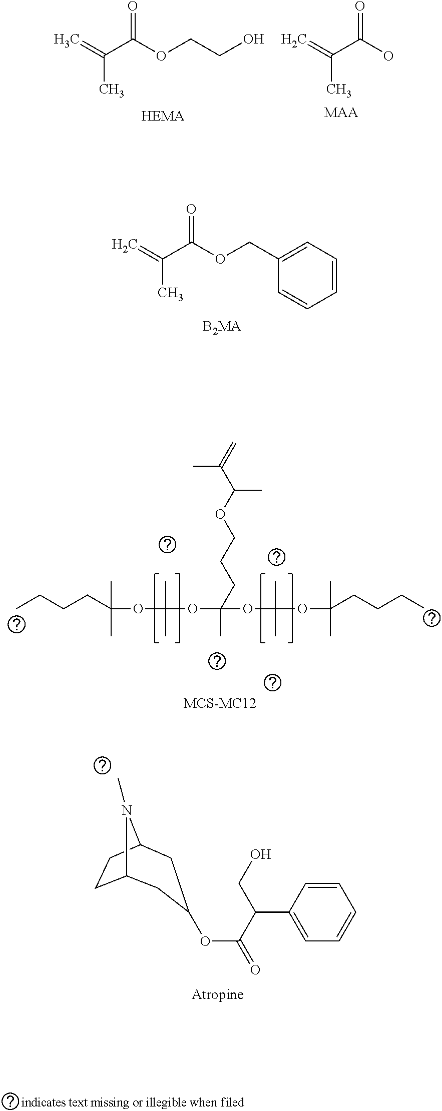

### Example 2—Preparation of Hydrogels of the Invention

Precursor hydrogel discs dried at 37° C. for 24 h were placed in 10-mL glass vials containing 5 mL of atropine sulfate loading solution at 1 mg/mL in distilled water or NaCl 0.9% medium (isotonic with physiological fluids).

The amount of atropine sulfate loaded by the hydrogels after 48 h is shown in FIG. 1 for each hydrogel in distilled water (left bars) and NaCl 0.9% medium (right bars).

The amount of atropine loaded was calculated as the maximum amount released since no atropine was detected during the extraction procedure carried out immediately after the release experiment.

The amount of atropine in the loading medium was quantified after half-dilution of the samples with NaCl 0.9% using a Waters HPLC (Autosampler Waters 717, Waters Controller 600, Photodiode Detector 996, Milford, MA, USA) equipped with a C18 column (Waters XSelect HSS T3, 3.5 μm, 4.6×150 mm) and operated with the Empower2 software. The analysis was carried out by isocratic elution using a mobile phase of acetonitrile:buffer 30:70 v/v at a flow rate of 1 mL/min for 6 min. (injection volume was 50 μL, column temp: 30° C.). The calibration curve was performed with standard solutions of atropine in NaCl 0.9% (0.625-60 μg/mL) and the absorbance measured at 210 nm. Retention times were ~3.7 min. The experiments were carried out in quadruplicate.

### Example 3—Atropine Release Experiments

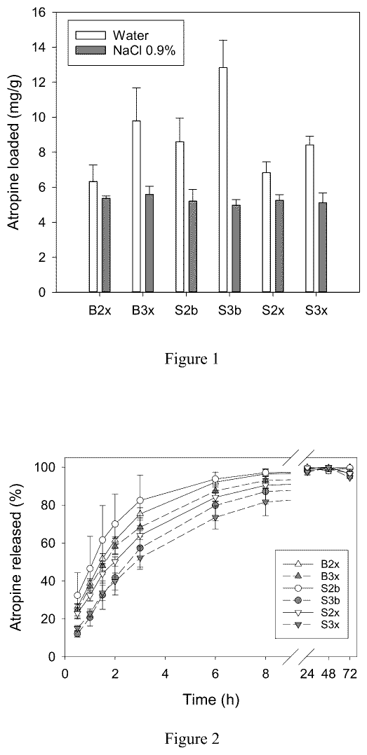

Precursor hydrogel discs dried at 37° C. for 24 h were placed in 10-mL glass vials containing 5 mL of atropine sulfate loading solution at 1 mg/mL in distilled water or NaCl 0.9% medium. After 48 h loading time, the discs were retrieved from the vials and rinsed with NaCl 0.9%. The rinsed discs were placed in 10 mL glass vials containing 6 mL of NaCl 0.9%. The experiments were carried out protected from light at 36° C. and 180 rpm for 24 h in an incubating shaker (Incubator 1000, Heidolph, Germany). Aliquots (300 μL) of the release medium were taken at times 30 min, 1 h, 2 h, 3 h, 6 h, 8 h, 24 h, 48 h and 72 h and replaced with the same volume of NaCl 0.9% fresh medium and atropine sulfate quantified by HPLC as described in Example 2. The amount of atropine sulfate released for each hydrogel in NaCl 0.9% medium are shown in FIG. 2.

As can be observed, results showed a sustained release of atropine for at least 8 h, in stark contrast to the marked immediate release profiles reported in the prior art for a large number of immersion-loaded hydrogels (see Hui et al. In vitro release of two anti-muscarinic drugs from soft contact lenses. Clinical Ophthalmology, 11, (2017), 1657; FIG. 2). The hydrogels of the present invention are therefore highly suited to overnight use.

### Example 4—Atropine Loading and Release Experiments Using Lower Loading Concentration and Sterilization

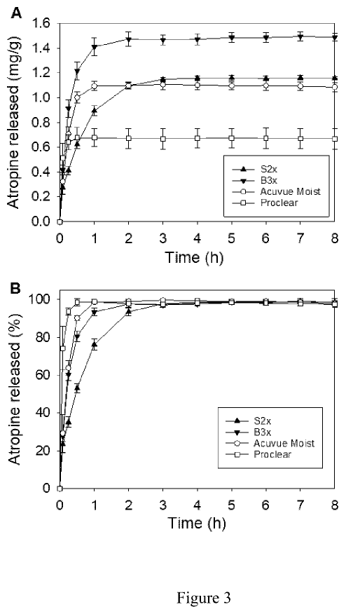

Experiments were also carried out to confirm that controlled release is also observed at lower atropine concentrations. Precursor hydrogel CLs S2x and B3x (0.1 mm thickness) dried at 37° C. for 24 h were placed in 10-mL glass vials containing 5 mL of atropine sulfate loading solution at 0.2 mg/mL in NaCl 0.9% medium. Immediately after, the vials were sealed and sterilized by steam heat (121° C., 20 min; Raypa Steam Sterilizer, Terrassa, Spain). After sterilization, the vials were kept at room temperature (25° C.) and 180 rpm for 3 days (Incubator 1000, Heidolph, Germany). The CLs were retrieved from the vials and rinsed with NaCl 0.9%. The rinsed CLs were placed in 10 mL glass vials containing 6 mL of NaCl 0.9%. The experiments were carried out at 36° C. and 180 rpm for 24 h in an incubating shaker (Incubator 1000, Heidolph, Germany). Aliquots (300 μL) of the release medium were taken and atropine sulfate quantified by HPLC as described in Example 2. The percentage released at each time point was calculated assuming that the percentage released at 24 h was 100%. For comparison purposes, commercially available Acuvue Moist and Proclear CLs were washed, dried and used for the loading and release experiments applying the same protocol as for S2x and B3x, and it was found that CLs according to the present invention attained a more controlled release of atropine than the commercial ones, the latter resulting in 100% atropine released in less than one hour. The amount of atropine sulfate released for each CL in NaCl 0.9% medium is shown in FIG. 3.

### Example 5—In Vivo Atropine Release Experiments

In vivo experiments using male New Zealand white rabbits were performed to evaluate the capacity of the developed CLs to provide a sustained release of atropine in vivo and facilitate its access to the different ocular tissues. The animal species was chosen regarding its extensive use in ophthalmology research due to the similarity between rabbit and human eyes.

### Animals Distribution and Stabling

All in vivo experiments were carried out following the Association for Research in Vision and Ophthalmology (ARVO) Statement for the Use of Animals in Ophthalmic and Vision Research and the European Directive 2010/63/EU. Male New Zealand white rabbits (age approx. 3 months and 3.5±0.6 Kg weight) were included for all in vivo experiments. All animals were stabled, for at least one week before the beginning of the experiments, in a light-controlled room with 12 h light-dark cycles inside individual cages with total access to food and water at 19° C. and 60% relative humidity. The animals were divided into two different groups: one group with animals treated with atropine-loaded CLs prepared from the precursor hydrogel S2x that were loaded in 0.2 mg/mL atropine sulfate solution as described in Example 4 (CLs group, n=4) and another one with animals treated with a single eye drop (50 μL) of 0.2 mg/mL atropine sulfate in NaCl 0.9% solution (eye drops group, n=4). The animals were euthanized after 6 h of the beginning of the experiment. To minimize the effects of subjective bias, each experiment was carried out in three days and the rabbits were randomly assigned to each assay day and treatment. All experiments started at 8:30-9:00 a.m. No animals or data were discarded and no adverse effects were detected.

i. In Vivo Release of Atropine

The experiments were carried out in the same way for both CLs and eye drops treatments, treating the right eyes and leaving the left ones as control for all animals. In the CLs groups, dried precursor hydrogel CLs were obtained, sterilized and loaded as described in Example 4. On the day of the test, the corresponding CLs were removed from the loading solution, rinsed with sterile saline solution (Avizor sterile saline unidose 5 mL) and placed on the right eye cornea below the nictitating membrane without local anesthesia. The rabbits were kept in rabbit restrainers with continuous monitoring to avoid CLs loss. The eyes of the rabbits were closed every 15 min for 1 min to prevent drying of the CL on the ocular surface. At the end of the experiment, remaining atropine in the CLs was extracted with MilliQ® water (1 mL, 36° C., 180 rpm, 8 h) and quantified by HPLC, as described above.

In the eye drops group, a single drop (50 μL) of sterilized by filtration (Polyethersulphone (PES) syringe filter 0.22 μm, Filter-Lab®, Barcelona, Spain) atropine solution (0.2 mg/mL in NaCl 0.9%) was gently instilled in the lower conjunctival sac of the right eye using a micropipette.

A VX75 slit-lamp (Luneau Technology, Chartres, France) was used to observe the ocular surface of all rabbits at 0 h, 1 h and 6 h, and the irritation score was assessed following the Draize criteria [Draize, J. H. (1944) J. Pharmacol. Exp. Ther., 82, 377-390]. The pupil diameter of both eyes of all animals was also monitored at times 0 h, 1 h, 4 h and 6 h. Samples of tear fluid were collected before (t=0 h) and after treatment (t=5 min, 15 min, 30 min, and every hour for 6 h) using Schirmer test strips placed in the tarsal conjunctiva of the inferior lid for 10 s with closed eyes to avoid the reflex secretion associated with blinking. Tear volume collected was calculated checking the millimeters of wetted strip.

### Atropine Levels in Tear Fluid

After collection, the Schirmer test strips were cut into small pieces and placed in 1.5 mL Eppendorf® tubes containing 150 μL of NaCl 0.9%. The tubes were vortexed for 1 min to ensure atropine extraction and the strips removed. Samples were kept in the fridge at 4° C. and protected from light until UPLC analysis. Before UPLC analysis all samples were diluted 1.5 times with acetonitrile and mixed using an Automated Liquid Handling System, Caliper Zephyr, 3 cycles of 50 μL at 78 μL/s. The plate was then centrifuged at 3,700 rpm and 4° C. for 30 min. The quantification of atropine was carried out using a Waters Acquity UPLC H-Class coupled with a Xevo TQD MS System fitted with a BEH C18 column (1.7 μm 2.1×50 mm, Waters) at a flow rate of 0.6 mL/min and 35° C. The analysis was performed by gradient elution using water+0.1% formic acid as solvent A, and Acetonitrile+0.1% formic acid as solvent B. The gradient program used was as follows: 0-0.1 min. 5% B, 0.1-1.0 min. 5-100% B, 1.0-2.0 min. 100% B, 2.0-2.1 min. 100-95% B, and 2.1-2.5 min. 5% B. Electrospray ionization (ESI) was run in positive mode with a source temperature of 150° C. and a desolvation temperature of 500° C. Capillary voltage was set to 3 kV and the cone voltage was set to 45 V. Desolvation gas flow was 900 L/h and cone gas flow was set to 50 L/h. The compound of interest was monitored in multiple reaction monitoring (MRM) mode. 290.221>124.166 m/z transition was quantified.

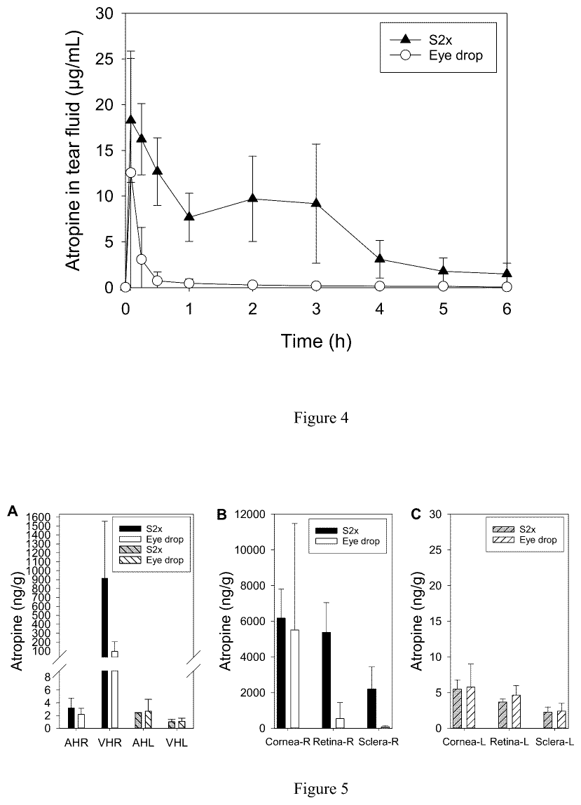

The in vivo release profiles of atropine in tear fluid are shown in FIG. 4. No atropine was detected for the samples of the left eyes. The highest concentration of atropine in the rabbit tear fluid was observed 5 min post-administration 18.29±6.79 μg/mL for S2x. In the eye drops group, the highest concentration of atropine in tear fluid was also obtained 5 min post-administration (12.56±13.31 μg/mL), but here, the concentration rapidly decreased after this point being zero after ~2 h post-administration.

### Atropine Ocular Distribution

At the end of the experiments, all rabbits were euthanized by intravenous injection of 0.75 mL/Kg of propofol and 0.5 mL/Kg of pentobarbital sodium. Immediately after, the aqueous humor of both eyes was directly extracted from the anterior chamber using a 25 G needle. Then, the eyes were enucleated and cornea, crystalline lens, vitreous humor, retina and sclera were separated. All different ocular tissues were kept in 1.5 mL Eppendorf® tubes to which different volumes of NaCl 0.9% were added (500 μL for cornea and crystalline lens, 200 μL for retina and 800 μL for sclera). The tubes were kept overnight in the fridge at 4° C. under soft stirring and then protein denaturation process was carried out as described in Pereira-da-Mota el at. (2022), Journal of Controlled Release, 348, 431-443. The supernatants were collected and kept at 4° C. until UPLC analysis as described above. The extraction and protein denaturation methods used have shown to reproducibly recover more than 99% atropine present in the samples.

The capacity of the atropine released to permeate through and accumulate into the anterior and posterior ocular tissues was also evaluated for both eyes of all rabbits. Atropine was detected in aqueous humor (AH), cornea, vitreous humor (VH), retina and sclera after 6 h treatment with eye drops and S2x CLs. S2x CLs led to significantly higher amounts of atropine in ocular tissues compared to the eye drops solution, especially for retina and sclera tissues (FIG. 5), because of the higher and sustained levels of atropine in the tear fluid provided by the developed CLs preventing clearance of atropine from the ocular surface through the nasolacrimal drainage or due to blinking.

1. Hydrogel comprising:
a polymer network, wherein the polymer network:
comprises acrylic monomeric units in an amount of at least 90% by weight with respect to the total weight of the polymer network; and
is cross-linked;
atropine or a salt thereof,
wherein the acrylic monomeric units comprised in the polymer network comprise anionic acrylic monomeric units; and
wherein the acrylic monomeric units comprised in the polymer network comprise aromatic acrylic monomeric units or comprise silicon oxide acrylic monomeric units, wherein the aromatic groups comprised in said aromatic acrylic monomeric units comprise no N heteroatoms.

2. Hydrogel according to claim 1, wherein the aromatic groups comprised in the aromatic acrylic monomeric units comprise no N, O or S heteroatoms.

3. Hydrogel according to claim 2, wherein the aromatic groups comprised in the aromatic acrylic monomeric units comprise no heteroatoms.

4. Hydrogel according to claim 1, wherein the polymer network does not comprise molecular imprinting sites for atropine or a salt thereof.

5. Hydrogel according to claim 1, wherein the atropine comprised in the hydrogel is in the form of atropine sulfate.

6. Hydrogel according to claim 1, comprising the atropine or salt thereof in an amount of at least 0.1% by weight with respect to the total weight of the hydrogel.

7. Hydrogel according to claim 1, wherein the acrylic monomeric units comprised in the polymer network comprise an amount of at least 90% by weight of monofunctionalized acrylic monomeric units, and an amount of between 0.01% and 10% by weight of bifunctionalized acrylic monomeric units with respect to the total weight of acrylic monomeric units.

8. Hydrogel according to claim 1, wherein the acrylic monomeric units comprised in the polymer network comprise an amount of between 0.001% and 5%, by weight of anionic acrylic monomeric units with respect to the total weight of acrylic monomeric units.

9. Hydrogel according to claim 1, wherein the acrylic monomeric units comprised in the polymer network comprise an amount of between 2% and 15%, by weight of aromatic acrylic monomeric units with respect to the total weight of acrylic monomeric units

10. Hydrogel according to claim 1, wherein the acrylic monomeric units comprised in the polymer network comprise an amount of between 2% and 65%, by weight of silicon oxide acrylic monomeric units with respect to the total weight of acrylic monomeric units.

11. Hydrogel according to claim 1, wherein the acrylic monomeric units comprised in the polymer network comprise:
an amount of at least 90% by weight of monofunctionalized acrylic monomeric units; and an amount of between 0.01% and 10% by weight of bifunctionalized acrylic monomeric units with respect to the total weight of acrylic monomeric units;
an amount of between 0.1% and 5% by weight of carboxylic acid anionic acrylic monomeric units with respect to the total weight of acrylic monomeric units;
an amount of between 2% and 15% by weight of aromatic acrylic monomeric units with respect to the total weight of acrylic monomeric units;
an amount of between 2% and 15% by weight of silicon oxide acrylic monomeric units with respect to the total weight of acrylic monomeric units.

12. A hydrogel comprising:
a polymer network, wherein the polymer network:
comprises acrylic monomeric units in an amount of at least 90% by weight with respect to the total weight of the polymer network; and
is cross-linked;
wherein the acrylic monomeric units comprised in the polymer network comprise anionic acrylic monomeric units; and
wherein the acrylic monomeric units comprised in the polymer network comprise aromatic acrylic monomeric units or comprise silicon oxide acrylic monomeric units, wherein the aromatic groups comprised in said aromatic acrylic monomeric units comprise no N heteroatoms.

13. (canceled)

14. Hydrogel according to claim 12, wherein the aromatic groups comprised in the aromatic acrylic monomeric units comprise no heteroatoms.

15. Hydrogel according to claim 12, wherein the polymer network does not comprise molecular imprinting sites for atropine or a salt thereof.

16. (canceled)

17. Method for preparing a hydrogel, comprising the steps of:
a) Providing a precursor hydrogel comprising a polymer network, wherein the polymer network:
comprises acrylic monomeric units in an amount of at least 90% by weight with respect to the total weight of the polymer network, wherein said acrylic monomeric units comprise anionic acrylic monomeric units; and wherein said acrylic monomeric units comprise aromatic acrylic monomeric units or silicon oxide acrylic monomeric units, wherein the aromatic groups comprised in said aromatic acrylic monomeric units comprise no N heteroatoms;
is cross-linked;
b) Providing an atropine or salt thereof solution;
c) Combining the precursor hydrogel of step a) and the atropine solution of step b).

18. Method according to claim 17, wherein the aromatic groups comprised in the aromatic acrylic monomeric units comprised in the polymer network of the precursor hydrogel provided in step a) comprise no N, O or S heteroatoms.

19. Method according to claim 17, wherein the aromatic groups comprised in the aromatic acrylic monomeric units comprised in the polymer network of the precursor hydrogel provided in step a) comprise no heteroatoms.

20. Method according to claim 17, wherein the polymer network comprised in the precursor hydrogel provided in step a) does not comprise molecular imprinting sites for atropine or a salt thereof.

21. Method according to claim 17, wherein the atropine or salt thereof solution of step b) is an atropine sulfate solution wherein the concentration of atropine sulfate is between 0.1 and 20 mg/mL.

22. Method according to claim 17, wherein the combining of step c) is performed by immersing the precursor hydrogel in the atropine or salt thereof solution.

23. (canceled)

24. (canceled)
